(19) United States
(12) Patent Application Publication
(10) Pub. No.: US 2025/0084452 A1
(43) Pub. Date: Mar. 13, 2025

(54) BIOLOGICAL INDICATOR FOR DETERMINING THE EFFICACY OF A STERILIZATION PROCESS AND METHODS OF USE

(71) Applicant: TERRAGENE LLC, Spring, TX (US)

(72) Inventors: Adrián Jesús ROVETTO, Alvear (AR); Esteban LOMBARDÍA, Alvear (AR); Pablo RAVASI, Alvear (AR); Carlos Ernesto RODRIGUEZ, Alvear (AR)

(21) Appl. No.: 18/962,747

(22) Filed: Nov. 27, 2024

Related U.S. Application Data
(63) Continuation-in-part of application No. 17/367,224, filed on Jul. 2, 2021.

(51) Int. Cl. C12Q 1/22

(52) U.S. Cl. CPC C12Q 1/22

(57) ABSTRACT

A biological indicator for determining the efficacy of a sterilization process, and its methods of use. The biological indicator comprises a set of microbial spores, at least one sensor molecule, and a culture medium, the sensor molecule being capable of yielding an optically detectable signal when the sensor molecule is not in a inactivated state due to the sterilization process, and a different optically detectable signal when the sensor molecule is in a inactivated state after the sterilization process.

### FIELD OF THE INVENTION

The present invention refers to biological indicators suitable for determining the outcome of a sterilization process, such as a sterilization process, a reductive sterilization process, or a thermal sterilization process using dry or wet heat.

### BACKGROUND OF THE INVENTION

Sterilization processes are aimed at providing surfaces or objects (e.g. laboratory or medical devices, instruments or utensils) that are free of viable forms of life. Such processes are used extensively in contexts like the healthcare industry or a wide range of scientific research activities.

A central aspect of such processes is the ability to determine whether the sterilization process was successful, in order to ensure the required conditions of use of the sterilized material and/or surface. To this end, several methods are employed with varying degrees of quality.

Biological indicators known in the art generally involve subjecting a known amount of microbial spores, such as bacterial spores, to a sterilization process along with the target material and/or surface. Immediately after the process is complete, tests are carried out in order to probe the presence of remaining living and/or viable microorganisms. If these tests yield negative results, then it may be determined that the sterilization was effective.

More specific biological indicators include probing the occurrence of certain biochemical reactions that are known to indicate the presence of viable forms of life. Such biochemical reactions relate to enzymatic and/or catalytic activity commonly found in microbial life or color change in a dye.

Patent application US 2015/0159192 A1 discloses a method of determining the success of a sterilization process comprising the use of an isolated enzyme or the microorganism to which such enzyme is endogenous or expressed by genetic engineering. Indicator enzymes according to this disclosure are enzymes found commonly in spore forming microorganisms such as beta-D-glucosidase. After the indicator has been exposed to a sterilization process, an enzymatic activity test is carried out in order to assess the effectiveness of the sterilization.

Other biological indicators are based on the use of genetically engineered microorganisms capable of expressing specific reporter genes.

Patent application WO 2018/071732 A1 discloses a biological indicator making use of genetically engineered microorganisms capable of expressing reporter genes that are suitable to be screened for fluorescence (e.g. reporter genes suitable for expression of fluorescent proteins). After the indicator has been subjected to the sterilization process, it is screened for optically detectable signals, thus evidencing the presence or absence of viable microorganisms.

Similarly, patent application WO 2017/185738 A1 discloses a biological indicator based on the use of spores from genetically engineered microorganisms expressing specific fluorescent reporter genes. After the sterilization process, the indicator is screened for optically detectable signals, in order to assess the presence of viable microorganisms.

Other biological indicators known in the prior art comprise providing a genetically engineered microorganism suitable for expression of specific enzymes, in order to screen enzymatic activity of said specific enzyme after sterilization.

Patent application US 2017/0292143 A1 discloses genetically engineered microorganisms suitable for expression of a specific enzyme (e.g. β-lactamase) which is able to hydrolyze a fluorogenic compound designed to emit fluorescence by hydrolysis. The optically detectable fluorescent signal therefore indicates the presence of viable microorganisms.

Other biological indicators employ the screening of surrogate proteins selected from proteins critical for the growth of infectious agents, and pathogenic or immunogenic proteins.

Patent application US 2017/0283847 A1 discloses a biological indicator based on the screening for a defined surrogate protein after a sterilization process. The disclosed method requires procedures such as Western Blot analysis in order to assess the presence of the target protein.

Other biological indicators of similar characteristics also employ genetically modified microorganisms or mutant and/or labeled proteins and/or enzymes in order to enable an effective screening after sterilization, such as disclosed by CA 2667698 C, U.S. Pat. No. 9,717,812 B2, EP 2456882 B1, U.S. Pat. No. 10,047,334 B2, US 20140370535 A1 and JP 2014-060947 A.

The biological indicators of the prior art generally rely on complex, costly and significantly time-consuming procedures for both production and use, affecting the overall costs of the biological indicator per se and of sterilization processes in general. Biological indicators based on such procedures demand considerable incubation and/or readout time, which also represents a significant expense in time and resources. Biological indicators that rely solely on screening enzymatic activity are limited by the generally low stability of the employed enzyme. Inherent characteristics of enzymes, like their structure and catalytic activity, have a negative effect on the overall stability of the indicator and the effectiveness of the system. Moreover, longer periods of time are needed in order to screen enzymatic activity.

Therefore, the need remains for a biological indicator that is both reliable and cost-effective, capable of reducing incubation and/or readout times, and without involving costly procedures and/or requirements.

Additionally, it is desirable that the indicator does not require development stages comprising Western Blot analysis, protein array analysis, magnetic separation analysis, mass spectrometry analysis, peptide analysis, chromatography analysis, nor gas chromatography analysis, which require specialized equipment and are considerably time-consuming.

On the other hand, the fact that the biological indicator does not depend on enzymatic reactions to indirectly determine the outcome of a sterilization process provides more reliable results. Enzymatic reactions are complex physical and chemical phenomena that require a wide range of favorable conditions in order to take place. For example, and as is known to a person skilled in the art, enzymatic activity depends greatly on the structure and nature of an enzyme active site. Even a small variation in any of the characteristics of such active sites, or in the environmental conditions around the enzyme, may have significant consequences in the enzyme ability to properly carry out the reaction. The chances of such variations entail serious potential flaws for a biological indicator reliant on enzymatic reactions, as these might incorrectly indicate the success of a sterilization.

### BRIEF DESCRIPTION OF THE INVENTION

The present invention relies only on the results of two simple and straightforward tests, namely an optically detectable signal test (e.g. a fluorescence intensity test, an absorbance test, etc.) and a colorimetric test, rendering the overall process significantly more time-and cost-effective in relation to the prior art.

In a first aspect, the present invention relates to a self-contained biological indicator for determining the efficacy of a sterilization process, employing both an immediate optically detectable signal detection and a colorimetric test after a certain incubation time.

It is therefore an object of the present application a device for determining the efficacy of a sterilization process, comprising:
a) a set of microbial spores,
b) at least one sensor molecule,
c) a culture medium, and
d) a pH indicator or colorimetric component for extended readout confirmation.

The at least one sensor molecule is capable of yielding an optically detectable signal, e.g., a fluorescence signal, an absorbance signal, a reflectance signal and the like, when the at least one sensor molecule is not in a inactivated state due to the sterilization process and is further capable of yielding a different optically detectable signal, such as a signal with different intensity, depending on the tridimensional structure, the oxidation state or the presence of the at least one sensor molecule and the polarity of the environment, when the at least one sensor molecule is in a inactivated state after the sterilization process. Advantageously, this difference is independent of a catalytic activity of the at least one sensor molecule and the culture medium being brought into contact with the set of microbial spores. This difference is indicative of a successful sterilization process, e.g. a decrease in the optically detectable signal (i.e. between the optically detectable signal yielded by the sensor molecule in a non-inactivated state and the optically detectable signal yielded by the sensor molecule in a inactivated state) is indicative of a successful sterilization process.

As used herein, the term “sensor molecule” refers to a chemical entity which can be used to sense the efficacy of a sterilization process, as described in detail herein. This term may refer to both individual or groups of molecules, as well as to molecular complexes or structures, such as those formed by fluorophores interacting with proteins, peptides, saccharides, quenchers or “anti-fade” molecules acting on fluorophores, as disclosed in further detail herein below.

As used herein, the term “inactivated” or “inactivation” when referring to a sensor molecule should be understood to a state of said molecule wherein the spatial or three-dimensional conformation or configuration or chemical bonding structure of said molecule is such that it yields a substantially different optically detectable signal (e.g. a fluorescence signal) as compared to the optically detectable signal yielded by the molecule in its “non-inactivated” or “natural” state (i.e. before having been exposed to a sterilization process). In specific embodiments, the sensor molecule is no longer capable of yielding an optically detectable signal, (e.g. a fluorescence signal) when the sensor molecule is in its inactivated state. In other embodiments, the optically detectable signal is substantially different (e.g. it has a different intensity, a different wavelength or wavelength distribution, etc.). In the specific case where the molecule is a protein, “inactivated” refers to a structural conformation in which the protein's native three-dimensional structure is altered or disrupted through the application of external physical or chemical factors, such as those involved in sterilization processes, commonly referred to as “denatured”. For other sensor molecules (such as peptides, fluorophores, quenchers or “anti-fade” molecules), the term “inactivated” may refer to the result of “degradation”, “oxidation” or “reduction” of the sensor molecule, whereby the sensor molecule is in a “degraded”, “oxidized” or “reduced” state, or to any other terms related to a substantial change in conformation or configuration or chemical bonding structure as understood by a person having ordinary skill in the art.

In the context of the present invention, the difference in the optically detectable signal may be measured immediately after the sterilization process, such as in less than 5 min, preferably less than 1 min after the sterilization process. Even more preferably, it may be measured, 1 s, 3 s, 5 s or 7 s after the sterilization process.

The culture medium is capable of inducing growth of any viable microbial life present after the sterilization process, and comprises the colorimetric component being capable of undergoing an optically detectable color change in the presence of microbial growth.

In embodiments of the invention, the sterilization process is a process employing a chemical agent selected from an oxidizing agent and a reducing agent; or a physical agent selected from dry heat, wet heat, steam, UV-radiation, and gamma-radiation.

In embodiments of the invention, the sterilization process is a process employing an oxidizing agent selected from ozone, oxygen, hydrogen peroxide, chlorine dioxide, and sulfuric acid, or mixtures and combinations thereof. In other embodiments, the sterilization process is a process employing formaldehyde. The above-mentioned sterilization processes are not meant to limit to the scope of the invention.

In an embodiment of the invention, the set of microbial spores in a) are bacterial spores.

In a preferred embodiment of the invention the set of microbial spores in a) are bacterial spores selected from the group comprising B. atrophaeus, B. subtilis, G. stearothermophilus, and B. pumilus.

In a preferred embodiment of the present invention, the microbial spores are embedded in a carrier.

In another preferred embodiment of the present invention, the sensor molecule is also embedded in the carrier.

In another embodiment of the invention, the sensor molecule comprises a fluorophore selected from (E)-Stilbene, (Z)-Stilbene, 1-Chloro-9,10-bis(phenylethynyl)anthracene, 2-Chloro-9,10-bis(phenylethynyl)anthracene, 2-Chloro-9,10-diphenylanthracene, 3-Hydroxyisonicotinaldehyde, 5,12-Bis(phenylethynyl)naphthacene, 6-Carboxyfluorescein, 7-Aminoactinomycin D, 8-Anilinonaphthalene-1-sulfonic acid, 9,10-Bis(phenylethynyl)anthracene, Acridine orange, Acridine yellow, Acriflavine, Alexa Fluor dyes, Auramine-rhodamine stain, ATTO fluorophores, Benzanthrone, Bimane, Bisbenzimide, BODIPY dyes, Brilliant cresyl blue, BUV dyes, Calcein, Carboxyfluorescein diacetate succinimidyl ester, Carboxyfluorescein succinimidyl ester, Coumarin, Cresyl violet, Cyanine dyes, DAPI, Dichlorofluorescein, Dil, Diketopyrrolopyrrole dye, DiOC6, Diphenylhexatriene, DyLight dyes, DY dyes, EDANS, Eosin, Eosin B, Eosin Y, Epicocconone, Erythrosine, Ethidium bromide, FIASH-EDT2, Fluo-3, Fluo-4, FluoProbes, Fluorescein, Fluorescein isothiocyanate, Fura-2, Fura dyes, Gallocyanin, GelGreen, GelRed, Heptamethine dyes, Hoechst stain, IAEDANS, iFLuor dyes, Iminocoumarin, Indian yellow, Indo-1, Indocyanine green, Infracyanine green, Laurdan, Lucifer yellow, Merocyanine, mFluor dyes, NBD-TMA, Nile blue, Nile red, Pacific Blue, Pacific Green, Pacific Orange, Perylene, Phloxine, Phycobilin, Phycoerythrobilin, Prodan (dye), Propidium iodide, Pyranine, Reichardt's dye, Resazurin, Rhodamine, Rhodamine dyes, Rhodamine 123, Rhodamine 6G, Rhodamine B, RiboGreen, Rubrene, Seminaphtharhodafluor, Squaraine dye, Sulforhodamine 101, Sulforhodamine B, SYBR Gold, SYBR Green I, SYBR Safe, SYTO dyes, Tetraphenyl butadiene, Tetrasodium tris(bathophenanthroline disulfonate)ruthenium(II), Texas Red, Titan yellow, TSQ, Umbelliferone, Violanthrone, YOYO-1, Europium compounds, Terbium compounds, and Indocyanine green.

In another embodiment, the sensor molecule comprises an “anti-fade” molecule acting on a fluorophore. Anti-fade reagents are one of the main ingredients added to mounting mediums in fluorescence microscopy to reduce or prevent photobleaching of fluorescent dyes. Most anti-fade reagents are reactive oxygen species scavengers. Three of the most used anti-fade agents used to prevent photobleaching are p-Phenylenediamine (PPD), n-Propyl gallate (NPG), and 1,4-Diazabicyclo-octane (DABCOJ). Thus, in an embodiment, the sensor molecule is selected from p-Phenylenediamine (PPD), n-Propyl gallate (NPG), and 1,4-Diazabicyclo-octane (DABCOJ). Other anti-fade agents that can be used include L-Ascorbic acid, Trolox (3,4-Dihydro-6-hydroxy-2,5,7,8-tetramethyl-2H-1-benzopyran-2-carboxylic acid), 1,4-diazabicyclo[2.2.2]octane, Glutathione and Mowiol 4-88.

In another embodiment the sensor molecule comprises a quencher of a fluorophore. The quencher can be any one of the known fluorescence quenchers, preferably selected from iodide, acrylamide, succinimide, pyridine, imidazole, and triethylamine. The quencher can be made to break down during the sterilization process, such that the fluorescence intensity emitted by the sample increases due to reduced inactivation of the fluorophore by the quencher.

The anti-fade molecule or the quencher can degrade in the presence of a chemical agent, for example an oxidizing agent or a reducing agent; or in the presence of a physical agent, for example dry heat, wet heat, steam, UV-radiation, or gamma-radiation.

An anti-fade molecule or a quencher can act upon a fluorophore selected from (E)-Stilbene, (Z)-Stilbene, 1-Chloro-9,10-bis(phenylethynyl)anthracene, 2-Chloro-9,10-bis(phenylethynyl)anthracene, 2-Chloro-9,10-diphenylanthracene, 3-Hydroxyisonicotinaldehyde, 5,12-Bis(phenylethynyl)naphthacene, 6-Carboxyfluorescein, 7-Aminoactinomycin D, 8-Anilinonaphthalene-1-sulfonic acid, 9,10-Bis(phenylethynyl)anthracene, Acridine orange, Acridine yellow, Acriflavine, Alexa Fluor dyes, Auramine-rhodamine stain, ATTO fluorophores, Benzanthrone, Bimane, Bisbenzimide, BODIPY dyes, Brilliant cresyl blue, BUV dyes, Calcein, Carboxyfluorescein diacetate succinimidyl ester, Carboxyfluorescein succinimidyl ester, Coumarin, Cresyl violet, Cyanine dyes, DAPI, Dichlorofluorescein, Dil, Diketopyrrolopyrrole dye, DiOC6, Diphenylhexatriene, DyLight dyes, DY dyes, EDANS, Eosin, Eosin B, Eosin Y, Epicocconone, Erythrosine, Ethidium bromide, FlAsH-EDT2, Fluo-3, Fluo-4, FluoProbes, Fluorescein, Fluorescein isothiocyanate, Fura-2, Fura dyes, Gallocyanin, GelGreen, GelRed, Heptamethine dyes, Hoechst stain, IAEDANS, iFLuor dyes, Iminocoumarin, Indian yellow, Indo-1, Indocyanine green, Infracyanine green, Laurdan, Lucifer yellow, Merocyanine, mFluor dyes, NBD-TMA, Nile blue, Nile red, Pacific Blue, Pacific Green, Pacific Orange, Perylene, Phloxine, Phycobilin, Phycoerythrobilin, Prodan (dye), Propidium iodide, Pyranine, Reichardt's dye, Resazurin, Rhodamine, Rhodamine dyes, Rhodamine 123, Rhodamine 6G, Rhodamine B, RiboGreen, Rubrene, Seminaphtharhodafluor, Squaraine dye, Sulforhodamine 101, Sulforhodamine B, SYBR Gold, SYBR Green I, SYBR Safe, SYTO dyes, Tetraphenyl butadiene, Tetrasodium tris(bathophenanthroline disulfonate)ruthenium(II), Texas Red, Titan yellow, TSQ, Umbelliferone, Violanthrone, YOYO-1, Europium compounds, Terbium compounds, and Indocyanine green.

In another embodiment of the invention, the sensor molecule is a fluorescent sensor protein selected from the group comprising green fluorescent proteins (GFPwt, EGFP, SFGFP, Emerald, avGFP, T-Saphire), blue fluorescent proteins (Sirius, Azurite, EBFP, EBFP2, mKalama1, TagGFP), cyan fluorescent proteins (ECFP, Cerulean, CyPet, mTurquoise2, SCFP), yellow fluorescent proteins (YFP, Citrine, Venus, YPet, SYFP, Topaz, mAmetrina), red fluorescent proteins (tdTomato, mPlum, DsRed, mCherry, mStrawberry, mRaspberry, mRuby) and orange fluorescent proteins (mOrange, mKO and mOrange2).

In another embodiment, the sensor molecule is selected from fluorescein-labeled peptides (FITC), cyanine-containing peptides, e.g. Cy3 or Cy5, Rhodamine (TRITC)-derived peptides, Peptides derived from fluorescent proteins, and peptides with environment-activatable sensors (pH, redox).

In addition, chimeric proteins of these fluorescent proteins, as well as chimeric proteins comprising a fluorescent protein fused to a non-fluorescent protein such as fibrin, elastin, casein, collagen, actin, keratin, albumin and enzymes like lysozyme, amylase, lipase, pepsin, glucosidase, phosphatase, galactosidase, chymotrypsin and lipase may also be used, but only due to their structural features, not in relation to their catalytic activity.

In another embodiment, the sensor molecule comprises an oligonucleotide that has a fluorophore on one terminus and a quencher on the other terminus. The oligonucleotide possesses a partially self-complementary sequence, such that it forms a stem-loop structure when free in solution, keeping the fluorophore and quencher labels in close proximity and leading to low fluorescence emission. The oligonucleotide sequence is chosen so that it can hybridize with an intended target sequence in a different DNA molecule, forming a duplex with higher stability than the stem-loop structure. The opening of the DNA double strand, after a sterilization process, allows the oligonucleotide to hybridize with target DNA sequences, breaking the quenched stem-loop structure, increasing the fluorescence levels after this binding. The DNA molecule containing the target sequence may be part of the bacterial spore, or may be an exogenous component added to the formulation.

In an embodiment, the oligonucleotide can have a fluorophore selected from (E)-Stilbene, (Z)-Stilbene, 1-Chloro-9,10-bis(phenylethynyl)anthracene, 2-Chloro-9,10-bis(phenylethynyl)anthracene, 2-Chloro-9,10-diphenylanthracene, 3-Hydroxyisonicotinaldehyde, 5,12-Bis(phenylethynyl)naphthacene, 6-Carboxyfluorescein, 7-Aminoactinomycin D, 8-Anilinonaphthalene-1-sulfonic acid, 9,10-Bis(phenylethynyl)anthracene, Acridine orange, Acridine yellow, Acriflavine, Alexa Fluor dyes, Auramine-rhodamine stain, ATTO fluorophores, Benzanthrone, Bimane, Bisbenzimide, BODIPY dyes, Brilliant cresyl blue, BUV dyes, Calcein, Carboxyfluorescein diacetate succinimidyl ester, Carboxyfluorescein succinimidyl ester, Coumarin, Cresyl violet, Cyanine dyes, DAPI, Dichlorofluorescein, Dil, Diketopyrrolopyrrole dye, DiOC6, Diphenylhexatriene, DyLight dyes, DY dyes, EDANS, Eosin, Eosin B, Eosin Y, Epicocconone, Erythrosine, Ethidium bromide, FlAsH-EDT2, Fluo-3, Fluo-4, FluoProbes, Fluorescein, Fluorescein isothiocyanate, Fura-2, Fura dyes, Gallocyanin, GelGreen, GelRed, Heptamethine dyes, Hoechst stain, IAEDANS, iFLuor dyes, Iminocoumarin, Indian yellow, Indo-1, Indocyanine green, Infracyanine green, Laurdan, Lucifer yellow, Merocyanine, mFluor dyes, NBD-TMA, Nile blue, Nile red, Pacific Blue, Pacific Green, Pacific Orange, Perylene, Phloxine, Phycobilin, Phycoerythrobilin, Prodan (dye), Propidium iodide, Pyranine, Reichardt's dye, Resazurin, Rhodamine, Rhodamine dyes, Rhodamine 123, Rhodamine 6G, Rhodamine B, RiboGreen, Rubrene, Seminaphtharhodafluor, Squaraine dye, Sulforhodamine 101, Sulforhodamine B, SYBR Gold, SYBR Green I, SYBR Safe, SYTO dyes, Tetraphenyl butadiene, Tetrasodium tris(bathophenanthroline disulfonate)ruthenium(II), Texas Red, Titan yellow, TSQ, Umbelliferone, Violanthrone, YOYO-1, Europium compounds, Terbium compounds, and Indocyanine green on one of its terminus.

In an embodiment, the oligonucleotide can have a quencher selected from BHQ-0, BHQ-1, BHQ-2, BHQ-3, dabcyl, TAMRA, QSY 7, QSY 9, QSY 21, QSY 35, ElleQuencher, Iowa Black, Eclipse in one of its terminus.

The opening of the DNA double strand can happen after a sterilization process employing a chemical agent selected from an oxidizing agent and a reducing agent; or a physical agent selected from dry heat, wet heat, steam, UV-radiation, and gamma-radiation.

In another embodiment, the oligonucleotide is inactivated by the sterilization process and can hybridize to a double-stranded DNA, single stranded DNA or RNA. This hybridization allows the fluorescence readout to stabilize over time. Depending on the sequence of the oligonucleotide, it can be established as a sensor molecule directly, without the use of a nucleic acid for hybridization. In this way, the self-hybridizing structure of the oligonucleotides labeled with a fluorophore can be used as a sensor for the sterilizing agent. The opening of the oligonucleotide structure changes and the fluorescence levels rise because the molecular beacon cannot self-hybridize in the new condition after the sterilization process.

In a preferred embodiment of the present invention, the culture medium is contained in a breakable ampoule.

In another embodiment of the invention, the culture medium components comprise bacteriological peptone, yeast extract and L-valine.

In a preferred embodiment of the present invention, the culture medium comprises a colorimetric component.

In an embodiment of the present invention, the colorimetric component of the culture medium is selected from a group comprising bromocresol purple, bromocresol green, phenol red, thymol blue, bromophenol blue, bromothymol blue, 6-chloro-3-indoxyl-alpha-D-glucopyranoside, 5-bromo-4-chloro-3-indolyl α-D-glucopyranoside, 6-chloro-3-indoxyl-beta-D-galactopyranoside, 5-bromo-4-chloro-3-indolyl β-D-galactopyranoside, 5-bromo-4-chloro-3-indoxyl phosphate.

In a yet more preferred embodiment of the present invention, the sensor molecule and the microbial spores are mixed together as a formulation in the carrier.

In a preferred embodiment of the present invention, the breakable ampoule is made of a material with low thermal expansion coefficient, preferably borosilicate glass.

In a second aspect of the invention, another object is a method of use of the previously described biological indicator of the invention, said method of use generally comprising:
a) placing the biological indicator along with a target material to be sterilized within a sterilizer,
b) carrying out a sterilization process,
c) placing the biological indicator in an incubator,
d) screening the biological indicator for immediate detectable changes in optically detectable signal intensity, while incubating the biological indicator in the incubator,
e) determining the efficacy of the sterilization process based on the screening carried out during step d),
f) extending the incubation of the biological indicator obtained in step d)
g) screening the incubated biological indicators obtained in step e) for an optically detectable color change, and
h) determining the efficacy of the sterilization process, according to optically detectable changes obtained in step g).

In an embodiment of the method of the present invention, in steps a) and b) the biological indicator is placed inside the sterilizer and alongside the target material.

In an embodiment of the method of the present invention, in step b) the sterilization process comprises employing a chemical agent selected from an oxidizing agent and a reducing agent; or a physical agent selected from dry heat, wet heat, steam, UV-radiation, and gamma-radiation.

In an embodiment of the method of the present invention, in step b) the sterilization process comprises employing an oxidizing agent selected from ozone, oxygen, hydrogen peroxide, chlorine dioxide, formaldehyde and sulfuric acid. Preferably, the sterilization process is a hydrogen peroxide sterilization process.

In a preferred embodiment of the method of the present invention, the incubator is selected from a Terragene Incubator Reader Bionova® (IC10/20FR or IC10/20FRLCD) or a MiniBio incubator. Other incubators such as BPH Photon Auto-Reader Incubator Bionova® and BHY Hyper Auto-Reader Incubator can also be used.

In another embodiment of the method of the present invention, in step c) the previously prepared available incubator is set at a temperature in the range of 30-70° C.

The temperature to which the previously prepared available incubator is set can be determined by a person having ordinary skill in the art to ensure the adequate growth of the microbial spores.

In embodiments wherein the set of microbial spores are bacterial spores selected from G. stearothermophilus, the incubator is set a temperature in the range of 55-65, preferably 60° C.

In embodiments wherein the set of microbial spores are bacterial spores selected from B. atrophaeus and B. subtilis, the incubator is set a temperature in the range of 30-40, preferably 37° C.

In an embodiment of the method of the present invention, in step c) the ampoule is crushed immediately before the biological indicator is placed within the incubator.

In an embodiment of the method of the present invention, during step d) the biological indicator is screened for changes in optically detectable signal intensity, immediately after sterilization and while starting incubation.

In an embodiment of the method of the present invention, during step g) the efficacy of the sterilization process is determined according to the results obtained from both steps d) and g). A positive result in any one of steps d) or f) indicates an incomplete or failed sterilization process.

### BRIEF DESCRIPTION OF THE FIGURES

FIG. 1 shows the fluorescence intensity of 25 μg of lyophilized ECFP after different injection times.

FIG. 2 shows the fluorescence intensity of 10 μg of lyophilized Cy3 after being exposed to steam sterilization cycles.

FIG. 3 shows a schematic side view of the biological indicator of the invention.

### DETAILED DESCRIPTION OF THE INVENTION

The biological indicator provided by the invention is a self-contained device, which means that the set of microbial spores, the one or more sensor molecules and the culture medium are contained within a single flexible container, which helps avoid possible contaminations due to manipulation. The culture medium is nonetheless separated from the set of microbial spores, within a breakable ampoule contained in the flexible container, due to the need to bring these two elements into contact only after the sterilization process is complete.

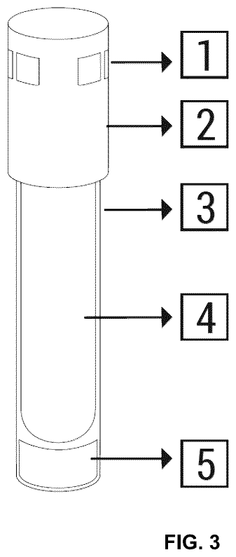

In an embodiment of the invention, the biological indicator comprises a flexible container as schematized in FIG. 3, comprising a cap 2 for sealing the flexible container, cap orifices 1, a container tube 3, an ampoule 4 containing culture medium and a carrier 5 containing the microbial spores.

In certain embodiments, the flexible container is a transparent polypropylene tube.

In another embodiment, the flexible container comprises a movable cap that may be pushed down in order to seal the container.

In other embodiments, the microbial spores are embedded in a carrier contained within the flexible container.

In certain embodiments, the microbial spores are embedded in a carrier made of a porous material, such as polypropylene fibers material, high-density polyethylene fibers, or in the polypropylene container itself. In other embodiments, the carrier comprises cellulose or is made of paper or cardboard.

The material of the carrier can be selected in accordance with the sterilization process, as is known to a person having ordinary skill in the art. Synthetic materials are preferred for sterilization processes employing chemical agents such as formaldehyde or hydrogen peroxide, while paper or other materials with cellulose fibers are preferred for steam or heat sterilization processes.

In a preferred embodiment, the high-density polyethylene fibers carrier is a sheet with a grammage of 55 to 80 g/m².

In an embodiment of the present invention, the ampoules are made of a fragile material with low thermal expansion coefficient, such as borosilicate glass, preferably glass, and contain 0.5 to 0.9 mL of culture medium.

In a preferred embodiment, the culture medium comprises 0.8 to 1.2 g/L of bacteriological peptone; 0.8 to 1.2 g/L of yeast extract and 0.4 to 0.6 g/L of L-valine.

In a preferred embodiment, the culture medium comprises 0.03 g/L of bromocresol purple indicator as a colorimetric component. Said culture medium has an adjusted pH between 7 and 9 preferably around 8.5. In a particular preferred embodiment, the pH is adjusted with sodium hydroxide.

In some embodiments, detection of a change in fluorescence intensity as a result of the inactivation of the at least one sensor molecule may be achieved by means of a device for detecting fluorescence intensity or fluorimeter.

In a preferred embodiment of the invention, the fluorimeter is integrated to the incubator, in order to minimize handling and movement of the biological indicator. This way, the detection of a change in fluorescence intensity can be made directly in the same incubator, without requiring further steps to obtain a fluorescence readout.

In some embodiments, detection of a change in color as a result of a colorimetric test may be achieved by means of direct visual observation or by means of a camera with subsequent image analysis.

The at least one sensor molecule is also contained in the carrier.

While contained in the carrier with the spores, the at least one sensor molecule and the spores are subjected to the same conditions (oxidizing agent concentration, time, humidity and temperature) as the target material to be sterilized.

In preferred embodiments, the amount of sensor molecule present in the carrier is in the range of 5-100 μg, preferably 25 μg.

Said at least one sensor molecule is a fluorescent sensor protein selected from the group comprising green fluorescent proteins (GFPwt, EGFP, SFGFP, Emerald, avGFP, T-Saphire), blue fluorescent proteins (Sirius, Azurite, EBFP, EBFP2, mKalama1, TagGFP), cyan fluorescent proteins (ECFP, Cerulean, CyPet, mTurquoise2, SCFP), yellow fluorescent proteins (YFP, Citrine, Venus, YPet, SYFP, Topaz, mAmetrina), red fluorescent proteins (tdTomato, mPlum, DsRed, mCherry, mStrawberry, mRaspberry, mRuby) and orange fluorescent proteins (mOrange, mKO and mOrange2).

Chimeric proteins, i.e., fusion proteins, comprising any number of these fluorescent proteins or chimeric proteins comprising a fluorescent protein fused to a non-fluorescent protein such as fibrin, elastin, casein, collagen, actin, keratin, albumin and enzymes like lysozyme, amylase, lipase, pepsin, glucosidase, phosphatase, galactosidase, chymotrypsin and lipase can also be used. These non-fluorescent proteins are to be selected in accordance with their structural features, not in relation to their catalytic activity.

In another embodiment, the fluorescent sensor protein comprises at least one post-translational modification selected from glycosylation, lipidation, phosphorylation, acylation, alkylation, palmitoylation, isoprenylation, myristoylation, or glypiation.

The fluorescent sensor protein may be present in a free, non-bound state, or may be complexed to another molecule or macromolecule, such as proteins, lipids, saccharides, polysaccharides, DNA, RNA, among others. This complex may be sensitive to the sterilization conditions, releasing the fluorescent sensor protein as the process progresses.

Fluorescence in fluorescent proteins, such as GFP, is directly linked to a “properly folded” protein structure. In the case of GFP, in its native, i.e., non-denatured, structure, the fluorescent tripeptide formed by serine 65, tyrosine 66 and glycine 67 has limited movement and is excluded from the bulk of water. Only when these fluorescent GFP proteins are denatured does the chromophore increase its rotational freedom and is further attacked by water molecules, leading to a quenching of the fluorescence phenomenon.

Oxidizing agents such as ozone, oxygen, hydrogen peroxide, chlorine dioxide, formaldehyde and sulfuric acid are highly reactive and predominantly target the cysteine residues of proteins. When any of these oxidizing agents is present in sufficiently high concentrations, the stress to which exposed macromolecules are subjected causes irreversible damage, resulting in denaturation of the protein tridimensional structure.

In embodiments of the present invention, the at least one sensor molecule is sensitive to chemical agents, for example oxidizing agents or reducing agents. Its tridimensional structure is closely related to conditions of the chemical agent sterilization process, such as exposition and concentration. Time of exposure to said conditions is also an important factor influencing the tridimensional structure of the sensor molecule.

In embodiments of the present invention, the at least one sensor molecule is a protein sensitive to oxidizing agents in general and hydrogen peroxide in particular. Its tridimensional structure is closely related to conditions of the hydrogen peroxide sterilization process, such as exposition and concentration. Time of exposure to said conditions is also an important factor influencing the tridimensional structure of the sensor molecule.

In another embodiment, the at least one sensor molecule to be used in the present invention is a protein sensitive to physical agents, for example dry heat, wet heat, steam, UV-radiation, or gamma-radiation. Its tridimensional structure is closely related to conditions of the physical agent sterilization process, such as exposition time. Time of exposure to said conditions is also an important factor influencing the tridimensional structure of the sensor molecule

In particular embodiments, the sensor molecule may be synthesized by microorganisms, such as genetically engineered microorganisms that express specific fluorescent macromolecules.

Non-limiting examples of genetically engineered microorganisms that synthetize this sensor molecule include B. atrophaeus, B. subtilis, G. stearothermophilus, and B. pumilus. These microorganisms are generally modified with a replicative plasmid capable of expressing the sensor molecule during the spore formation process. In this way, the sensor molecule will be a part of the spore proteins and will be in contact with the oxidizing agent during the sterilization process. Said at least one sensor molecule is at least one fluorescent sensor protein selected from the group comprising green fluorescent proteins (GFPwt, EGFP, SFGFP, Emerald, avGFP, T-Saphire), blue fluorescent proteins (Sirius, Azurite, EBFP, EBFP2, mKalama1, TagGFP), cyan fluorescent proteins (ECFP, Cerulean, CyPet, m Turquoise2, SCFP), yellow fluorescent proteins (YFP, Citrine, Venus, YPet, SYFP, Topaz, mAmetrina), red fluorescent proteins (tdTomato, mPlum, DsRed, mCherry, mStrawberry, mRaspberry, mRuby) and orange fluorescent proteins (mOrange, mKO and mOrange2).

In another embodiment, the sensor molecule is added exogenously and is therefore not synthesized by the bacteria nor is it the result of their metabolism.

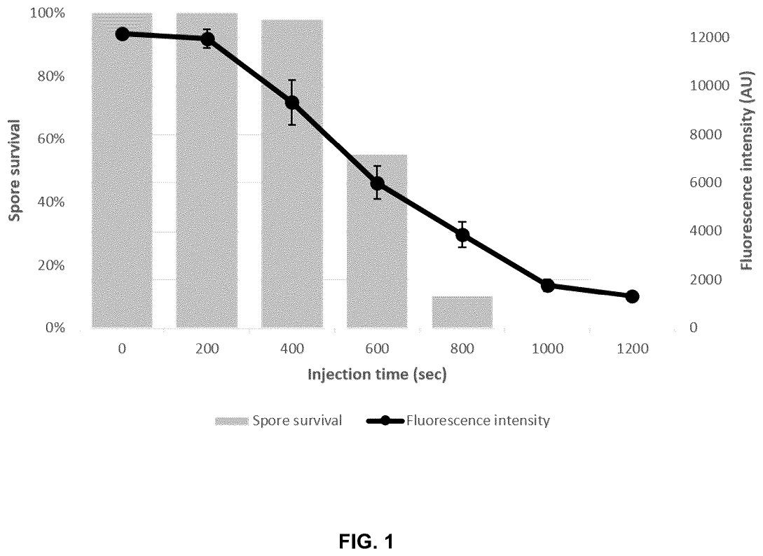

FIG. 1 shows the fluorescence intensity of 25 μg of ECFP after different injection times using a 5% v/v concentration of hydrogen peroxide in a CASP 50 flash device. ECFP was inoculated in the spore carrier, along with a population of 10⁶ Geobacillus stearothermophilus ATCC® 7953 bacterial spores. Immediately after the exposure, the inoculum was resuspended in 0.5 mL of culture medium (1 g/L of bacteriological peptone; 1 g/L of yeast extract, 0.3 g/l of L-valine, 0.03 g/l of bromocresol purple indicator, buffer tris base 0.015% at pH 8.5). Fluorescence was measured during 7 seconds of incubation with a fluorimeter setting up wavelengths between 260-460 nm for excitation, more preferably, 420-450 nm for excitation and 470-490 nm for emission. 20 tubes per injection time were evaluated. Dots represent mean values. Bars around the dots represent the standard deviation. The non-sterilized condition is also shown. Samples were subsequently incubated at 60° C. for 7 days in a humidity chamber and checked for spore survival by visual inspection. Solid bars show spore survival rate (Positive Biological Indicators/Exposed Biological Indicators). With the increase of injection time, the measured fluorescence is lower and the proportion of positive indicators for visual inspection decreases.

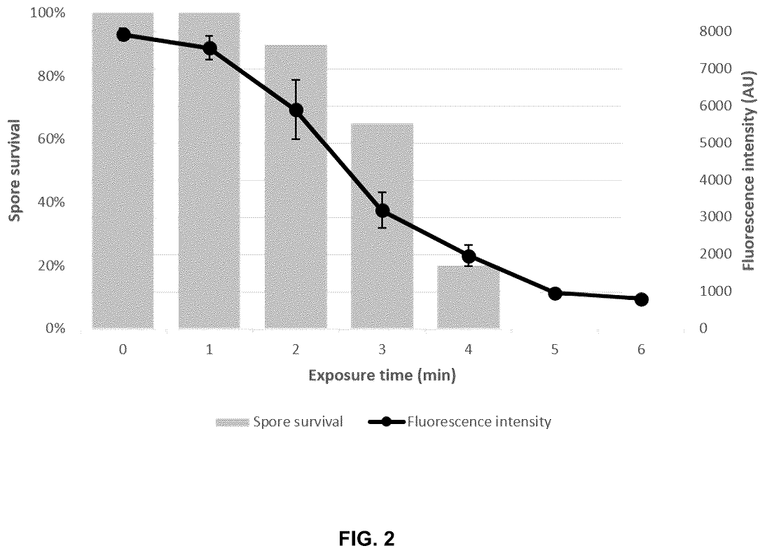

FIG. 2 shows the fluorescence intensity of 10 μg of lyophilized Cy3 after being exposed to steam sterilization cycles at 132° C. in a BIER (Biological Indicator Evaluator Resistometer). Cy3 was inoculated in the spore carrier, along with a population of 10⁶ Geobacillus stearothermophilus ATCC® 7953 bacterial spores. Immediately after the exposure, the inoculum was resuspended in 0.5 mL of culture medium (1 g/L of bacteriological peptone; 1 g/L of yeast extract, 0.3 g/l of L-valine, 0.03 g/l of bromocresol purple indicator, buffer tris base 0.015% at pH 8.5). Fluorescence was measured during 7 seconds of incubation with a fluorimeter set up to wavelengths between 488-532 nm for excitation, and 553-633 nm for emission. 20 tubes per exposure time were evaluated. Dots represent mean values. Bars around the dots represent the standard deviation. The non-sterilized condition is also shown. Samples were subsequently incubated at 60° C. for 7 days in a humidity chamber and checked for spore survival by visual inspection. Solid bars show spore survival rate (Positive Biological Indicators/Exposed Biological Indicators). With the increase of exposure time, the measured fluorescence is lower and the proportion of positive indicators for visual inspection decreases.

A person skilled in the art will recognize that changes in the structure of sensor molecules (e.g. inactivation, denaturation, oxidation, reduction, or degradation) exposed to sterilization agents are necessarily linked to the death of microorganisms that undergo the same exposure (since life is unsustainable for a microorganism when all or a significant majority of its constituent proteins are denatured). As such, a change in the structure and consequently loss of optically detectable signal of the sensor molecules of the present invention indicates the death of any living microorganism, including microbial spores.

In the biological indicator of the present invention, the inactivation of the sensor molecule is therefore directly correlated with the death of the microbial spores. Consequently, the change in the optically detectable signal resulting from the sensor molecule inactivation guarantees the prediction of death of the spore population contained in the biological indicator.

The reliance of the biological indicator on these particular phenomena allows for immediate or instantaneous results. Therefore, if desired, determination of the efficacy of a sterilization process is available instantly, without having to invest in lengthy incubation times or costly procedures. In this case, time for enzyme catalytic activity is not necessary. The detectable change rate in a protein tridimensional structure subjected to an oxidizing agent concentration can be measured instantly, as opposed to measuring an enzymatic reaction.

As previously mentioned, the sensor molecule may be a fluorescent sensor protein. Moreover, said fluorescent sensor protein may be a chimeric protein comprising a non-fluorescent protein, the non-fluorescent protein being selected from lysozyme, amylase, lipase, pepsin, glucosidase, phosphatase, galactosidase, chymotrypsin and lipase, and a fluorescent protein selected from the group comprising green fluorescent proteins (GFPwt, EGFP, SFGFP, Emerald, avGFP, T-Saphire), blue fluorescent proteins (Sirius, Azurite, EBFP, EBFP2, mKalama1, TagGFP), cyan fluorescent proteins (ECFP, Cerulean, CyPet, mTurquoise2, SCFP), yellow fluorescent proteins (YFP, Citrine, Venus, YPet, SYFP, Topaz, mAmetrina), red fluorescent proteins (tdTomato, mPlum, DsRed, mCherry, mStrawberry, mRaspberry, mRuby) and orange fluorescent proteins (mOrange, mKO and mOrange2). The fluorescent protein may be associated, i.e. fused, bound or linked, to the non-fluorescent protein by methods well known to one skilled in the art, such as by being produced as a transcriptional fusion by a modified expression host such as Escherichia coli.

The top of the container comprises materials such as polypropylene fibers or high-density polyethylene fibers, permeable to the sterilizer agent and a polypropylene cap with lateral openings. The device has been described in detail with reference to the accompanying FIG. 3.

Preferably, all elements are sterilized prior to assembly with ethylene oxide 800 mg/mL for 2 hours. Said elements are then assembled in sterile conditions, with the spore containing carrier being inoculated with the spores prior to assembly in sterile conditions as well.

The method of use of the biological indicator will be described as follows and will be illustrated by means of non-limiting examples.

The method of the present invention comprises:
a first step wherein the biological indicator is placed along with the target material to be sterilized within a sterilizer,
a second step wherein a sterilization process is carried out at 30-70° C.,
a third step wherein the biological indicator is placed in a previously conditioned incubator set at a temperature in the range of 30-70° C., preferably 55-65° C., more preferably 58-62° C., yet more preferably 60° C., and the ampoule is broken by crushing it immediately before the biological indicator is placed within the incubator,
a fourth step wherein the biological indicator is screened for optically detectable changes in optically detectable signal intensity, immediately after being placed in the previously conditioned incubator, while incubation of the biological indicator in the previously conditioned incubator is ongoing,
a fifth step wherein the efficacy of the sterilization process is determined according to the optically detectable signal intensity screening results of the fourth step,
a sixth step wherein the biological indicator is incubated for 24 hours-168 hours at a temperature in the range of 30-70° C., depending on the bacterial strain used,
a seventh step wherein the culture medium obtained in the sixth step is readout with a colorimetric component, screening for optically detectable changes in color 24-168 hours; and
a last step wherein the efficacy of the sterilization process is determined according to the results of the colorimetric screening of the seventh step.

In a particular embodiment of the method of the present invention, the biological indicator is placed along with the material to be sterilized. The biological indicator is located inside a sterilizer besides the target material to be sterilized, in order to ensure that it undergoes the same sterilization conditions as the target, related to the position and temperature within the sterilizer.

In another particular embodiment, the biological indicator is placed in the areas that are considered to be more inaccessible to the sterilizer agent. The sterilization process is subsequently carried out in the usual way. During this step, the cap of the biological indicator container is loose, i.e. the tube is not entirely sealed by the cap. In certain embodiments, the sterilization is carried out at 40-60° C. After the sterilization process is finished, the biological indicator is left to cool until it reaches room temperature. The incubator is previously conditioned for the readout and detection steps.

In a preferred embodiment, the incubator is capable of measuring the optically detectable signal by means of an integrated sensor. In a yet more preferred embodiment of the invention, the incubator is selected from the group consisting of a Terragene Incubator Reader Bionova® IC10/20FR, IC10/20FRLCD or a MiniBio incubator. The incubator is set to a temperature in the range of 30-70° C., depending on the bacterial strain used. Once the desired temperature is reached, the biological indicator cap is pressed to seal the tube, the ampoule is crushed, and the biological indicator is placed inside the previously prepared incubator. When the ampoule is crushed, the different components of the biological indicator are brought into contact. The ampoule should not be broken before the incubator reaches the desired temperature.

Immediately after being placed inside the incubator, the processed biological indicator yields an instantaneously detectable optically detectable signal. In general, the wavelengths used for excitation and emission will depend on the sensor molecule in the indicator.

In a particular embodiment of the invention, an effective sterilization is evidenced if fluorescence values change drastically (accordingly with the irreversible modification of the sensor molecule structure). If after 0 to 120 seconds of incubation an important change of optically detectable signal intensity is detected, it means that the sterilization was effective, sufficiently affecting the tridimensional structure of sensor molecules and changing the emission of optically detectable signal.

The incubation at a temperature in the range of 55-65° C., 58-62° C., or 60° C., depending on the bacterial strain used, continues to allow the microbial spores growth in order to perform the colorimetric test for 24-168 hours. In a particular embodiment of the invention, if the sterilization was ineffective, the microbial spores are viable and therefore grow, whereby the culture medium changes color. This phenomenon is due to the varying medium pH or a metabolic enzyme activity with colorimetric components caused by the growth of microbial life, as is well known in the art.

The sterilization efficacy will be determined both by the optically observed change in optically detectable signal and by the optically observed change in the culture medium. In a particular embodiment, a positive control should be used to observe and compare the change in color and the presence of optically detectable signal.

### EXAMPLES

### Example 1

The example was carried out using a CASP 50 Flash device, which allows to conduct sub lethal expositions in order to evaluate predictions of a biological indicator.

BIONOVA BT96 biological indicators were used for testing during the example along with the biological indicator of the present invention in order to obtain comparison data.

Fast readouts were obtained using an IC10/20FRLCD incubator. Extended readouts (7 days) were carried out in humidity chambers with 80% relative humidity at 60° C.

The culture medium contained in the ampoules of the biological indicator of the invention consisted of 1 g/L of bacteriological peptone; 1 g/L of yeast extract and 0.5 g/L of L-valine.

20 biological indicator of the invention and 20 control indicator tests were carried out for each particular exposure time.

Fluorescence readouts with the biological indicator of the invention were carried out within 0-20 seconds according to the method of the invention. Mean fluorescence values for 20 samples are shown in the table below. Ampoule contained bromocresol purple as colorimetric component of the culture medium.

Along with the ampoule, the carrier containing 2.5×10⁶ CFU of Geobacillus stearothermophillus ATCC 7953 spores and 5 μg of EBFP as fluorescent sensor protein was laid inside the tube. Exposure cycles with increasing injection times were conducted with a 5% v/v hydrogen peroxide concentration according to the method of the invention.

| | Mean | | | Positives 30 | Positives 7 |
|---|---|---|---|---|---|
| Injection | Fluorescence | | Positives 7 | min readout | days readout |
| Time | Instant | Standard | days | BIONOVA | BIONOVA |
| (seconds) | readout | Deviation | readout | BT96 | BT96 |
| 200 | 6531.15 | 267 | 20/20 | 20/20 | 20/20 |
| 300 | 5869.70 | 211 | 16/20 | 20/20 | 14/20 |
| 400 | 4489.55 | 191 | 4/20 | 16/20 | 7/20 |
| 500 | 3271.10 | 120 | 0/20 | 4/20 | 0/20 |
| 600 | 2654.90 | 135 | 0/20 | 0/20 | 0/20 |

### Example 2

Conditions of Example 1 were replicated, except for the variations indicated herein.

Ampoule contained bromocresol purple as colorimetric component of the culture medium.

Along with the ampoule, the carrier containing 2.5×10⁶ CFU of Geobacillus stearothermophillus ATCC 7953 spores and 50 μg of mCherry as fluorescent sensor protein was laid inside the tube. Exposure cycles with increasing injection times were conducted with a 5% v/v hydrogen peroxide concentration according to the method of the invention.

| | Mean | | | Positives 30 | Positives 7 |
|---|---|---|---|---|---|
| Injection | Fluorescence | | Positives 7 | min readout | days readout |
| Time | Instant | Standard | days | BIONOVA | BIONOVA |
| (seconds) | readout | Deviation | readout | BT96 | BT96 |
| 200 | 4921.15 | 173 | 20/20 | 20/20 | 20/20 |
| 300 | 3756.30 | 158 | 13/20 | 19/20 | 14/20 |
| 400 | 3117.80 | 141 | 7/20 | 12/20 | 5/20 |
| 500 | 2518.35 | 121 | 0/20 | 2/20 | 0/20 |
| 600 | 2036.50 | 103 | 0/20 | 0/20 | 0/20 |

### Example 3

Conditions of Example 1 were replicated, except for the variations indicated herein.

Ampoule contained 5-Bromo-4-chloro-3-indolyl β-D-galactopyranoside as colorimetric component of the culture medium.

Along with the ampoule, the carrier containing 2.5×10⁶ CFU of Geobacillus stearothermophillus ATCC 7953 spores and 100 μg of YFP as fluorescent sensor protein was laid inside the tube. Exposure cycles with increasing injection times were conducted with a 5% v/v hydrogen peroxide concentration according to the method of the invention.

| | Mean | | | Positives 30 | Positives 7 |
|---|---|---|---|---|---|
| Injection | Fluorescence | | Positives 7 | min readout | days readout |
| Time | Instant | Standard | days | BIONOVA | BIONOVA |
| (seconds) | readout | Deviation | readout | BT96 | BT96 |
| 200 | 9723.40 | 340 | 20/20 | 20/20 | 20/20 |
| 300 | 7658.65 | 331 | 19/20 | 20/20 | 12/20 |
| 400 | 5248.25 | 229 | 5/20 | 15/20 | 3/20 |
| 500 | 4671.20 | 249 | 0/20 | 6/20 | 1/20 |
| 600 | 3964.30 | 198 | 0/20 | 0/20 | 0/20 |

### Example 4

Conditions of Example 1 were replicated, except for the variations indicated herein.

Ampoule contained bromothymol blue was used as colorimetric component of the culture medium.

Along with the ampoule, the carrier containing 2.5×10⁶ CFU of Geobacillus stearothermophillus ATCC 7953 spores and 75 μg of ECFP as fluorescent sensor protein was laid inside the tube. Exposure cycles with increasing injection times were conducted with a 5% v/v hydrogen peroxide concentration according to the method of the invention.

| | Mean | | | Positives 30 | Positives 7 |
|---|---|---|---|---|---|
| Injection | Fluorescence | | Positives 7 | min readout | days readout |
| Time | Instant | Standard | days | BIONOVA | BIONOVA |
| (seconds) | readout | Deviation | readout | BT96 | BT96 |
| 200 | 6924.20 | 198 | 20/20 | 20/20 | 20/20 |
| 300 | 6094.90 | 300 | 17/20 | 17/20 | 10/20 |
| 400 | 5149.85 | 218 | 4/20 | 5/20 | 1/20 |
| 500 | 4836.25 | 231 | 0/20 | 0/20 | 0/20 |
| 600 | 4298.75 | 225 | 0/20 | 0/20 | 0/20 |

### Example 5

Conditions of Example 1 were replicated, except for the variations indicated herein.

Ampoule contained bromothymol blue was used as colorimetric component of the culture medium.

Along with the ampoule, the carrier containing 2.5×10⁶ CFU of Bacillus subtilis ATCC 35021 spores and 25 μg of mOrange as fluorescent sensor protein was laid inside the tube. Exposure cycles with increasing injection times were conducted with a 5% v/v hydrogen peroxide concentration according to the method of the invention.

| | Mean | | | Positives 30 | Positives 7 |
|---|---|---|---|---|---|
| Injection | Fluorescence | | Positives 7 | min readout | days readout |
| Time | Instant | Standard | days | BIONOVA | BIONOVA |
| (seconds) | readout | Deviation | readout | BT96 | BT96 |
| 200 | 7737.05 | 332 | 20/20 | 20/20 | 20/20 |
| 300 | 7249.25 | 346 | 14/20 | 20/20 | 13/20 |
| 400 | 6683.80 | 213 | 5/20 | 8/20 | 1/20 |
| 500 | 5944.70 | 195 | 0/20 | 2/20 | 0/20 |
| 600 | 5462.50 | 246 | 0/20 | 0/20 | 0/20 |

1. A biological indicator for determining the efficacy of a sterilization process, wherein said biological indicator comprises, in a single container:
a set of microbial spores;
at least one sensor molecule; and
a culture medium,
wherein the at least one sensor molecule being capable of yielding an optically detectable signal when the at least one sensor molecule is not in a inactivated state due to the sterilization process, the at least one sensor molecule being further capable of yielding a different optically detectable signal when the at least one sensor molecule is in a inactivated state after the sterilization process, said difference being independent of a catalytic activity of the at least one sensor molecule and the culture medium being brought into contact with the set of microbial spores,
wherein said culture medium being capable of inducing growth of any viable microbial life present after the sterilization process, and
wherein said culture medium comprising a colorimetric component being capable of undergoing an optically detectable color shift in the presence of microbial growth.

2. The biological indicator of claim 1, wherein the sterilization process is a process employing a chemical agent selected from an oxidizing agent and a reducing agent, and mixtures thereof; or a physical agent selected from dry heat, wet heat, steam, UV-radiation, and gamma-radiation.

3. The biological indicator of claim 2, wherein the sterilization process is a process employing dry heat, wet heat or steam.

4. The biological indicator of claim 2, wherein the sterilization process is a process employing UV-radiation or gamma-radiation.

5. The biological indicator of claim 1, wherein the set of microbial spores are bacterial spores.

6. The biological indicator of claim 3, wherein the bacterial spores are spores originating from bacteria selected from the group comprising B. atrophaeus, B. subtilis, G. stearothermophilus, and B. pumilus.

7. The biological indicator of claim 1, wherein the set of microbial spores is embedded in a carrier made of a porous material or in the container.

8. The biological indicator of claim 1, wherein the set of microbial spores is embedded in the container.

9. The biological indicator of claim 1, wherein the at least one sensor molecule is a complex selected from an “anti-fade” molecule acting on a fluorophore, a quencher acting on a fluorophore, a peptide labeled with a fluorophore, and an oligonucleotide comprising a fluorophore on one terminus and a quencher on the other terminus.

10. The biological indicator of claim 1, wherein the at least one sensor molecule is an oligonucleotide labeled with a fluorophore.

11. The biological indicator of claim 1, wherein the at least one sensor molecule comprises a fluorophore selected from (E)-Stilbene, (Z)-Stilbene, 1-Chloro-9,10-bis(phenylethynyl)anthracene, 2-Chloro-9,10-bis(phenylethynyl)anthracene, 2-Chloro-9,10-diphenylanthracene, 3-Hydroxyisonicotinaldehyde, 5,12-Bis(phenylethynyl)naphthacene, 6-Carboxyfluorescein, 7-Aminoactinomycin D, 8-Anilinonaphthalene-1-sulfonic acid, 9,10-Bis(phenylethynyl)anthracene, Acridine orange, Acridine yellow, Acriflavine, Alexa Fluor dyes, Auramine-rhodamine stain, ATTO fluorophores, Benzanthrone, Bimane, Bisbenzimide, BODIPY dyes, Brilliant cresyl blue, BUV dyes, Calcein, Carboxyfluorescein diacetate succinimidyl ester, Carboxyfluorescein succinimidyl ester, Coumarin, Cresyl violet, Cyanine dyes, DAPI, Dichlorofluorescein, DiI, Diketopyrrolopyrrole dye, DiOC6, Diphenylhexatriene, DyLight dyes, DY dyes, EDANS, Eosin, Eosin B, Eosin Y, Epicocconone, Erythrosine, Ethidium bromide, FLASH-EDT2, Fluo-3, Fluo-4, FluoProbes, Fluorescein, Fluorescein isothiocyanate, Fura-2, Fura dyes, Gallocyanin, GelGreen, GelRed, Heptamethine dyes, Hoechst stain, IAEDANS, iFLuor dyes, Iminocoumarin, Indian yellow, Indo-1, Indocyanine green, Infracyanine green, Laurdan, Lucifer yellow, Merocyanine, mFluor dyes, NBD-TMA, Nile blue, Nile red, Pacific Blue, Pacific Green, Pacific Orange, Perylene, Phloxine, Phycobilin, Phycoerythrobilin, Prodan (dye), Propidium iodide, Pyranine, Reichardt's dye, Resazurin, Rhodamine, Rhodamine dyes, Rhodamine 123, Rhodamine 6G, Rhodamine B, RiboGreen, Rubrene, Seminaphtharhodafluor, Squaraine dye, Sulforhodamine 101, Sulforhodamine B, SYBR Gold, SYBR Green I, SYBR Safe, SYTO dyes, Tetraphenyl butadiene, Tetrasodium tris(bathophenanthroline disulfonate)ruthenium(II), Texas Red, Titan yellow, TSQ, Umbelliferone, Violanthrone, YOYO-1, Europium compounds, Terbium compounds, and Indocyanine green.

12. The biological indicator of claim 1, wherein the at least one sensor molecule is a fluorescent protein selected from a green fluorescent protein, a blue fluorescent protein, a cyan fluorescent protein, a yellow fluorescent protein, a red fluorescent protein, an orange fluorescent protein, and combinations or chimeric proteins thereof.

13. The biological indicator of claim 1, wherein the at least one sensor molecule is a protein complexed to another molecule selected from a protein, a lipid, a saccharide, a polysaccharide, and a polynucleotide.

14. The biological indicator of claim 1, wherein the at least one sensor molecule is a chimeric protein comprising a non-fluorescent protein selected from lysozyme, amylase, lipase, pepsin, glucosidase, phosphatase, galactosidase, chymotrypsin and lipase and a fluorescent protein selected from the group comprising green fluorescent proteins (GFPwt, EGFP, SFGFP, Emerald, avGFP, T-Saphire), blue fluorescent proteins (Sirius, Azurite, EBFP, EBFP2, mKalama1, TagGFP), cyan fluorescent proteins (ECFP, Cerulean, CyPet, mTurquoise2, SCFP), yellow fluorescent proteins (YFP, Citrine, Venus, YPet, SYFP, Topaz, mAmetrina), red fluorescent proteins (tdTomato, mPlum, DsRed, mCherry, mStrawberry, mRaspberry, mRuby) and orange fluorescent proteins (mOrange, mKO and mOrange2).

15. The biological indicator of claim 1, wherein the at least one sensor molecule is selected from a fluorescein-labeled peptide (FITC), a cyanine-containing peptide, a rhodamine (TRITC)-derived peptide, a peptide derived from fluorescent proteins, and a peptides with environment-activatable sensors.

16. The biological indicator of claim 1, wherein the colorimetric component of the culture medium is selected from bromocresol purple, bromocresol green, phenol red, thymol blue, bromophenol blue, bromothymol blue, 6-chloro-3-indoxyl-alpha-D-glucopyranoside, 5-bromo-4-chloro-3-indolyl α-D-glucopyranoside, 6-chloro-3-indoxyl-beta-D-galactopyranoside, 5-bromo-4-chloro-3-indolyl β-D-galactopyranoside, 5-bromo-4-chloro-3-indoxyl phosphate and mixtures thereof.

17. The biological indicator of claim 1, wherein the culture medium is contained in a container separate from the microbial spores.

18. The biological indicator of claim 1, wherein the at least one sensor molecule and the microbial spores are mixed together as a formulation in the carrier.

19. The biological indicator of claim 1, wherein the at least one sensor molecule is embedded in a carrier within the biological indicator.

20. A method of determining the efficacy of a sterilization process, by means of a self-contained biological indicator, wherein said biological indicator comprises, in a single container, a set of microbial spores, at least one sensor molecule, and a culture medium,
wherein the at least one sensor molecule being capable of yielding an optically detectable signal when the at least one sensor molecule is not in a inactivated state due to the sterilization process, the at least one sensor moleculre being further capable of yielding a different optically detectable signal when the at least one sensor molecule is in a inactivated state after the sterilization process, said difference being independent of a catalytic activity of the at least one sensor molecule and the culture medium being brought into contact with the set of microbial spores,
wherein said culture medium being capable of inducing growth of any viable microbial life present after the sterilization process,
wherein said culture medium comprising a colorimetric component being capable of undergoing an optically detectable color shift in the presence of microbial growth, and
wherein the method comprises the steps of:
a) placing the biological indicator along with a target material to be sterilized within a sterilizer;
b) carrying out a sterilization process;
c) placing the biological indicator in an incubator;
d) screening the biological indicator for immediate detectable changes in fluorescence intensity, while incubating the biological indicator in the incubator;
e) determining the efficacy of the sterilization process based on the screening carried out during step d);
f) extending the incubation of the biological indicator obtained in step d);
g) screening the incubated biological indicators obtained in step e) for an optically detectable color change; and
h) determining the efficacy of the sterilization process, according to optically detectable changes obtained in step g).
